(19) United States
(12) Patent Application Publication
(10) Pub. No.: US 2003/0129678 A1
(43) Pub. Date: Jul. 10, 2003

(54) Methods, pharmaceutical formulations and kits for identification of subjects at risk for cancer

(76) Inventor: Constance Neely, Raleigh, NC (US)

Correspondence Address: MYERS BIGEL SIBLEY & SAJOVEC, PO BOX 37428, RALEIGH, NC, 27627, US

(21) Appl. No.: 10/316,423

(22) Filed: Dec. 11, 2002

Related U.S. Application Data
(63) Continuation of application No. 09/569,394, filed on May 12, 2000, now abandoned.
(60) Provisional application No. 60/134,276, filed on May 14, 1999.

(51) Int. Cl. G01N 33/574; A61K 38/21; A61K 31/739

(52) U.S. Cl. 435/7.23; 424/85.5; 514/54

(57) ABSTRACT

Subjects at risk for developing cancer may be identified by obtaining samples of diagnostic cells from the subjects and determining a measure of cytotoxicity of the cells, the measure of cytotoxicity correlating negatively with the risk of developing cancer. The development of cancer may be prevented in subjects determined to be at risk for developing cancer by administering priming and activating agents to the subject, by increasing the expression of A₁ adenosine receptors in cells of the subject, and increasing the affinity of cells of the subject for A₁ adenosine receptor ligands. The preventative and diagnostic methods of the present invention may be carried out with kits and pharmaceutical liposomal formulations.

### RELATED APPLICATIONS

This application is a continuation of U.S. patent application Ser. No. 09/569,394, filed May 12, 2000, which in turn claims the benefit of U.S. Provisional Application No. 60/134,276, filed May 14, 1999, which applications are incorporated herewith in their entirety.

### FIELD OF THE INVENTION

The present invention relates to methods, pharmaceutical compositions and kits useful in identifying subjects at risk for developing cancer, and additionally relates to methods, pharmaceutical compositions and kits useful in preventing the development of cancer in at-risk individuals.

### BACKGROUND OF THE INVENTION

Circulating monocytes derived from bone marrow promonocytes migrate into tissues where they differentiate into mature macrophages. Macrophages are a major component of the lymphoreticular infiltrate of tumors. Tumor-associated macrophages can be activated with immunomodulators and other biological agents to kill tumor cells. Andreesen, Res. Immunol. 144:291 (1993). The tumoricidal effect of macrophages is selective for tumor cells, is independent of tumor-specific antigens or transplantation antigens, and is dependent on direct cell-to-cell contact and the macrophage-to-tumor cell ratio. Whitworth, et al., Cancer and Metastasis Rev. 8:319 (1989). While the tumoricidal effect of macrophages appears to be unrelated to the resistance of the tumor cell to chemotherapy, it is also thought that under certain circumstances, tumor burden may exceed the destructive capability of available activated macrophages. Thus, the efficacy of activated macrophages to destroy tumor cells may be dependent on (1) the presence of a sufficient number of functioning, activated tumoricidal macrophages at the tumor site, and (2) recruitment of circulating monocytes which, following differentiation to macrophages, bind and destroy tumor cells. However, tumor-associated macrophages from mouse and human malignant tumors and their metastases have not been previously shown to be potent effectors of tumoricidal activity in the absence of stimulation. See Mantovani, Immunol. Today 13:265 (1992).

Several approaches to cancer treatment with activated monocytes and macrophages have been reported. Some of these approaches are based on the recognition that activated human blood monocytes and monocyte-derived macrophages possess cytotoxic effects against tumor cell lines when stimulated by various biological agents, including gamma interferon (IFN-γ), lipopolysaccharide (LPS), or granulocyte macrophage colony stimulating factor (GM-CSF). These approaches include activation of macrophages by local or systemic administration of bacterial products (including LPS), activation of circulating monocytes with intravenous liposomes containing immunomodulators (e.g., N-acetylmuramyl-L-analyl-D-isoglutamyl-L-analyl-2-(1′,2′-dipalmitoyl)-sn-glycerol-3′-phosphorylethylamide (MTP-PE)), and local or intravenous “adoptive cellular immunotherapy.” See, e.g., Whitworth, et al., Cancer and Metastasis Rev. 8:319 (1989); Salgaller and Lodge, J. Surg. Oncol. 68:122 (1998); Hennemann, et al., Cancer Immunol. Immunother. 45:250 (1998); Andreesen, et al., Cancer Detect. Prev 15:413 (1991); Eymard, et al, Eur. J. Cancer 32A: 1905 (1996); Faradji, et al., Cancer Immunol. Immunother. 33:319 (1991); Andreesen, et al., Cancer Res. 50:7450 (1990); Otto, et al., Eur. J. Cancer 32A: 1712 (1996). For example, methods of adoptive cellular immunotherapy with differentiated, tumor-cytotoxic and in vitro-generated macrophages have been developed. These methods were developed in order to overcome local factors affecting maturation or suppression of functional activation of tumor site-restricted macrophages. Following leukapharesis of a patient afflicted with cancer, autologous cells are reinfused into the patient with increasing doses and over several weeks. This method has been attempted both with and without prior treatment of the patient with intravenous immunomodulators (e.g., GM-CSF) to increase the yield of monocytes and macrophages. Prior to reinfusion into the patient, differentiation of the monocytes to macrophages is accomplished in vitro, followed by in vitro activation of the differentiated macrophages with immunomodulators or other biological agents, such as IFN-γ or LPS. In some cases, these autologous, differentiated, and activated tumor-cytotoxic macrophages are site-directed to the tumor and administered locally (e.g., intraperitoneally). Andreesen and Hennemann, Pathobiology 59:259 (1991). However, clinical responses to the treatment of human malignancies, with (1) intravenous or local administration of activated autologous macrophages; (2) intravenous liposomes containing immunomodulating agents; or (3) local or systemic treatment with immunomodulators or bacterial products have not been reported.

In subjects suffering from malignant cancer, the cytotoxic effect of the subjects' monocyte/macrophage cells is impaired (i.e., these cells lack spontaneous cytotoxic effects toward tumor cells). In one report, less than 25% of cancer patients possessed macrophages capable of killing their own tumor cells. Cameron, et al., Cancer 53:2053 (1984). It has also been shown that despite activation in vitro with LPS, monocyte/macrophage cells in patients with cancer lack cytotoxic effects toward both autologous and allogeneic tumor cells. See Cameron and O'Brien, Cancer 50:498 (1982); Cameron, et al., Cancer 53:2053 (1984); Key, Cancer Metastasis Rev. 2:75 (1983); Triozzi, et al., Bone Marrow Transplant. 18:47 (1996). This impairment may be a function of maturation or differentiation and/or responsiveness and function of these cells.

It has also been reported that following terminal differentiation, macrophages have a greater tumor cytotoxic effect than monocytes following stimulation with IFN-γ. Andreesen, et al., Cancer Res. 50:7450 (1990). In one particular study, peripheral blood monocytes obtained from normal donors or breast cancer patients were tested immediately after harvesting and activation; enhanced cytotoxicity was not observed. When monocytes were allowed to mature in vitro for 5 days, however, normal macrophages developed enhanced cytotoxicity while breast cancer patients' macrophages did not acquire enhanced cytotoxicity. Cameron and O'Brien, Cancer 50:498 (1982).

Cameron and O'Brien also reported that macrophages derived from more than 50% of patients with colon and hematological malignancies were cytotoxic for allogeneic tumor cells derived from a colon cell line. Id. In this report, cytotoxicity was measured in terms of the percentage of tumor cells killed by activated macrophages, and a cytotoxicity of greater than 10% was considered statistically significant. The percent cytotoxicity of activated macrophages from patients with colon cancer for allogeneic tumor cells ranged from 11 to 21%. Activated macrophages obtained from patients with breast and gynecological tumors were not cytotoxic for tumor cells derived from allogeneic breast and prostate cell lines. Moreover, a macrophage inhibitory factor was demonstrated in the plasma of patients with colon cancer with cytotoxic macrophages and in the plasma of patients with gynecological cancer with cytotoxic macrophages. Out of 66 patients studied with either breast, colon, gynecological or hematological cancers, 47 out of 50 patients' macrophages were incapable of killing allogeneic tumor cells in vitro either because they were unresponsive to activation or because of the presence of an inhibitory factor in their plasma. It was concluded that although some patients with cancer may possess macrophages that can be activated in vitro, they may be ineffective in vivo.

Triozzi, et al. report that in patients with metastatic cancers, peripheral blood stem cells were mobilized with GM-CSF. Bone Marrow Transplant. 18:47 (1996). Spontaneous monocyte/macrophage tumor cytotoxicity was not detectable, either before or after cryopreservation. However, cell cytotoxicity was inducible in vitro with IFN-γ against K562 cells with an effective monocyte/macrophage to tumor cell ratio of 20-10 to 1. The tumoricidal effect of these cells against the patients' own tumor cells was not tested. In another report, spontaneous monocyte-mediated cytotoxicity (SMMC) was severely depressed in patients with malignancies as compared with normal controls [7% (malignancies) vs. 43% (normal)]. Kleinerman et al., Lancet ii:1102 (1980). This same study showed that in seven patients with advanced ovarian cancer, treatment with cisplatin increased the SMMC at least three-fold. Id. In a third study, monocyte/macrophage tests, including macrophage precursor tests, antibody-dependent cellular cytotoxicity tests, spontaneous cellular cytotoxicity tests, and measurements of monocyte lysozyme activity were performed on monocytes from patients with breast, colon, head and neck, lung, and melanoma cancers and controls. Unger, et al., Cancer 51:669 (1983). Selected assays of peripheral blood monocyte function were abnormal in certain types of cancer patients and normal in others, and did not show consistent correlations with tumor type or stage. Id.

Various mouse and human tumors release factors that are chemotactic for the migration of monocytes. Monocyte chemotactic protein (MCP-1) is chemoattractive for monocytes but is inactive for lymphocytes and neutrophils. Montovani, et al., Immunol. Today 13: 265 (1992). MCP-1 is the human homologue of JE, a gene originally identified in mouse fibroblasts and a member of the superfamily of cytokines called chemokines. It interacts with a receptor that is coupled to a pertussis toxin-sensitive G protein to induce a rapid increase in intracellular free calcium. In addition to tumor cells, fibroblasts, smooth muscle cells, endothelial cells, and mononuclear cells themselves can produce MCP-1. Gene transfer of human MCP-1 into a mouse melanoma cell line has been reported. Bottazzi, et al., J. Immunol. 148:1280 (1992). After successful transformation, a prolongation of doubling time of the tumor cell lines and an increase in tumor associated macrophages was observed. However, there was no reduction in tumorigenicity.

LPS has been shown to activate macrophages to secrete cytokines, (including tumor necrosis factor-α (TNF-α), and to cause them to become tumoricidal Chen, et al., Curr. Topics in Microbiol Immunol. 181:169 (1992). The expression of mouse and human genies for cytokine expression, including TNF-α and interleukin-1β, are promoted by LPS activation of nuclear translocation of nuclear factor κB (NF-κB) in macrophages. Sweet and Hume, J. Leukoc. Biol. 60:8 (1996). Following priming with priming agents (e.g., phorbol myristoyl acetate (PMA), IFN-γ, f-met-leu-phe (fMLP), GM-CSF, lymphokines, muramyl dipeptide (MDP), muramyl tripeptide (MTP), and MTP-PE), macrophages can be fully activated by LPS. Priming is associated with an enhanced metabolic/oxidative burst and the release of various oxygen metabolites, including superoxide anion and H₂O₂. Hamilton and Adams, Immunol. Today 8:151 (1987). Moreover, priming induces macrophages to bind to tumor cells but, in the absence of activation, does not induce these cells to be fully cytotoxic for tumor cells. Adams and Hamilton, Ann. Rev. Immunol 2:283 (1984).

Endotoxin (i.e., LPS) binds to and activates A₁ adenosine receptors. Neely, et al., Am. J. Physiol. (Lung Cell Mol. Physiol.) 272:L353 (1997). A₁ adenosine receptors are coupled to a pertussis toxin-sensitive G protein, and activation of this receptor increases intracellular free calcium. Arend, et al., Am. J. Physiol. (Cell Physiol.) 255:C581 (1988). Moreover, human monocytes express A₁ adenosine receptors and oxidative stress increases A₁ adenosine receptor expression by NF-κB. Salmon, et al., J. Immunol. 151:2775 (1993); Nie, et al., Mol Pharmacol 53:663 (1998). It was recently reported that adenosine inhibits tumor cell growth of the tumor cell lines Nb2 lymphoma, K-562 leukemia, and LNCaP prostate carcinoma cells, while stimulating proliferation of normal murine bone marrow cells. Fishman, et al., Cancer Res. 58:3181 (1998).

In 1974, a strain of mice that is resistant to LPS was developed. Watson and Riblet, J. Exp. Med. 140:1147 (1974). Following treatment with intravenous LPS, macrophages from LPS-sensitive C3H/HeN mice were found to be tumoricidal. However, macrophages from LPS-resistant C3H/HeJ mice could not be activated for tumor cytotoxicity. Ruco and Meltzer, J. Immunol. 120:329 (1978). C3H/HEJ mice have a genetic defect that significantly reduces their ability to respond to LPS. Kraatz, et al., Shock 11:58 (1999). This hyporesponsiveness to LPS has been confirmed both in in vitro and in vivo studies. The precise nature of this molecular defect in these mice is unknown. The ability of these macrophages to respond to LPS is controlled by a single gene, Lps^{d}, which is located on chromosome 4. This defect is restricted to the inability of C3H/HeJ mice to respond to LPS; all other known functions of cells from these mice are normal. Some investigators believe this defect is related to an early signal transduction event and the ability to produce cytokines, such as TNF-α. However, the precise signal transduction defect is not known. Ruco and Meltzer, J. Immunol. 120:329 (1978); Sakagami, et al., Infect. Immun. 65:3310 (1997). Macrophages elicited from C3H/HeJ mice by Bacillus Calmette-Guerin (BCG) bind to tumor cells. However, only in C3H/HeN macrophages did LPS induce the expression of TNF-α. Adams and Hamilton, Ann. Rev. Immunol 2:283 (1984); Sweet and Hume, J. Leukoc. Biol. 60:8 (1996).

In spite of the foregoing, it has heretofore not been appreciated that a predictive relationship exists between the cytotoxicity of a subject's macrophages and monocytes against cancer cells, and the risk of the development of cancer in the subject. The tumoricidal effects of primed and activated macrophages and monocytes have not been examined in the context of developing diagnostic methods for determining a subject's risk of developing cancer.

### SUMMARY OF THE INVENTION

The present invention is based on the inventor's realization that a reliable predictor of a subject's likelihood or susceptibility to developing cancer is the measure of cytotoxicity of certain diagnostic cells (i.e., macrophages or monocytes) of the subject for target cancer cells. The measure of cytotoxicity may be an indirect measure, such as a measure of the number of A₁ adenosine receptors (A₁AR) produced by the diagnostic cells, or a measure of the affinity of the diagnostic cells for A₁ adenosine receptor specific ligands (i.e., a measure of the affinity of the A₁ARs present in the membranes of the diagnostic cells for A₁AR specific ligands), or a measure of the ability of the cells (i.e., the A₁ARs present in the membranes of the cells) to bind MCP-1 protein, or a measure of the ability of the cells (i.e., the A₁ARs present in the membranes of the cells) to bind annexins. Alternatively, the measure of cytotoxicity may be a functional measure of the cytotoxicity of the cell for cancer cells (i.e., a measure of the ability and efficiency of the A₁ adenosine receptors of the cell to couple to G proteins and signal-transduction-related pathways including protein kinase C, tyrosine kinase, phospholipase C, phospholipase D, phospholipase A₂, or NF-κB; a measure of the ability of the cell to induce the release of cytotoxic substances or cytokines, including interleukin-1, TNF-α, or thromboxane; or a measure of the ability of the cells to kill target cancer cells). The measure of cytotoxicity is negatively correlated to the risk of developing cancer; that is, the higher the cytotoxicity of the subject's own cells for cancer cells, the lower the risk of the subject developing cancer. The inventor has additionally discovered that methods of increasing the cytotoxicity of the diagnostic cells in those subjects found to be at-risk for developing cancer may actually prevent the development of cancer in those subjects. Moreover, the inventor has discovered that cloned or commercially available monocytes, macrophages, and precursors thereof that have increased cytotoxicity against tumor cells may be used to prevent the development of cancer in at-risk patients.

One particular advantage of the present invention relates to the unfortunate fact that by the time most cancers are detectable by current detection methods, they are large or widespread enough to have already caused significant morbidity in the subject. Accordingly, one advantage of the presently described diagnostic method for determining the risk of developing cancer is that preventative steps may be taken prior to the onset of disease. An advantage of the methods of preventing cancer disclosed herein is that the onset, spread, or recurrence of cancer may be partially or even completely prevented prior to the development of a cancer that is detectable (i.e., when the cancer does not yet exist or is not detectable by current detection methods, or when the cancer is in a microscopic, micrometastatic or microvascular state), or after a previously-existing cancer has been eradicated or “cured” by prior treatments.

Accordingly, a first aspect of the present invention is a method of determining a subject's risk for developing cancer, carried out by obtaining a sample of diagnostic cells (e.g., macrophages, monocytes, promonocytes, peripheral blood stem cells) from a subject; and then determining a measure of cytotoxicity of the diagnostic cells for target cancer cells, the measure of cytotoxicity correlating negatively with the risk for developing cancer. The measure of cytotoxicity may be an indirect measure of cytotoxicity or a functional measure of cytotoxicity.

A second aspect of the present invention is a method of preventing cancer in a subject at risk for developing cancer, carried out by administering to the subject a priming agent in an amount effective to prime macrophages of the subject. Optionally, an activating agent may also be administered to the subject.

A third aspect of the invention is a method of preventing cancer in a subject at risk for developing cancer, carried out by increasing the expression of A₁ adenosine receptors in the cells of the subject, the cells being selected from the group consisting of monocytes, macrophages, promonocytes and peripheral blood stem cells.

A fourth aspect of the invention is a method of preventing cancer in a subject at risk for developing cancer, carried out by increasing the affinity of the cells of the subject for A₁ adenosine receptor ligands, the cells being selected from the group consisting of monocytes, macrophages, promonocytes and peripheral blood stem cells.

A fifth aspect of the invention is a pharmaceutical liposomal formulation for the prevention of cancer in a subject determined to be at risk for developing cancer, comprising a priming agent and an activating agent encased in liposomes.

A sixth aspect of the invention is a diagnostic kit for determining a subject's risk for developing cancer comprising at least one reagent for determining the cytotoxicity of diagnostic cells of the subject, and printed instructions for assessing the subject's risk for developing cancer, packaged together in a container.

A seventh aspect of the invention is a kit for preventing cancer in a subject determined to be at-risk for the development of cancer, comprising at least one reagent selected from the group consisting of reagents for increasing A₁ adenosine receptor expression in macrophages, monocytes, promonocytes and peripheral blood stem cells, reagents for increasing binding of A₁ adenosine receptor ligands to macrophages, monocytes, promonocytes and peripheral blood stem cells, reagents for increasing binding of MCP-1 protein for macrophages, monocytes, promonocytes and peripheral blood stem cells, priming agents and activating agents; and printed instructions for administering the at least one reagent to the subject, packaged together in a container.

The foregoing and other aspects of the present invention are explained in detail in the specification set forth below.

### BRIEF DESCRIPTION OF THE DRAWINGS

FIG. 1 is a graphical illustration showing that following priming with PMA (1 μm) and activation with 2-chloro-N⁶-cyclopentyladenosine (CCPA) (lam), macrophages from LPS-sensitive mice (IC-21, ATCC) produce 50% cytotoxicity of mouse B-16 melanoma cells versus 20% cytotoxicity produced by macrophages from LPS-resistant mice (WBC264-9C, ATCC).

FIG. 2 is a graphical illustration showing that following priming with PMA (1 μM) and activation with an A₁ AR agonist, CCPA (1 μM), mouse macrophages (IC-21, ATCC) are cytotoxic for mouse P815 mastocytoma tumor cells.

FIG. 3 is a graphical illustration showing that following priming with PMA (1 μm) and activation with CCPA (1 μm), human macrophages (SC hematopoietic cell line, ATCC and a healthy volunteer) are cytotoxic to A375 human melanoma tumor cells.

FIG. 4 is a graphical illustration showing that in saturation experiments, A₁ adenosine receptors are expressed in macrophages from both LPS-sensitive mice (IC-21, ATCC) and LPS-resistant mice (WBC264-9C, ATCC) [Bₘₐₓ 420 (LPS-sensitive) versus Bₘₐₓ 425 (LPS-resistant)]; however, the affinity (K_{d}) of A₁ adenosine receptors for a highly selective A₁ adenosine receptor ligand, ¹²⁵ I BWA844U, is greater in LPS-sensitive mouse macrophages than LPS-resistant mouse macrophages [K_{d}=11.83 (LPS-sensitive) macrophages versus K_{d}=22.47 (LPS-resistant) macrophages].

FIG. 5 is a graphical illustration showing that in saturation experiments, treatment of human macrophages (SC, ATCC) with cisplatin (2.5 μM) for 24 hours increased A₁ adenosine receptor expression by approximately three-fold.

### DETAILED DESCRIPTION OF THE INVENTION

The present invention provides a generalized strategy for identifying subjects at risk for developing cancer, and for preventing cancers in subjects identified as being at risk for developing cancer. An at-risk subject is any individual who is believed to be at a higher risk than the general population for developing cancer.

The present invention is suitable for both medical and veterinary uses. Suitable subjects include, but are not limited to, mammalian and avian subjects. More preferred subjects are mammalian subjects such as humans, monkeys, pigs, cattle, dogs, horses, cats, sheep, and goats. The most preferred subjects are human subjects.

The term “cancer” as used herein is intended to encompass cancers of any origin, including both tumor-forming and non-tumor forming cancers. The term “cancer” has its understood meaning in the art, for example, an uncontrolled growth of tissue that has the potential to spread to distant sites of the body (i.e., metastasize). As used herein, the term “cancer cell” is also intended to encompass those cells referred to as “precancerous,” i.e., cells that contain mutated or damaged DNA or other components, which mutations or damage are likely to cause the cell to develop into a cancer cell. Exemplary cancers include osteosarcomas, angiosarcomas, fibrosarcomas and other sarcomas; leukemias; sinus tumors; ovarian, uretal, bladder, prostate and other genitourinary cancers; colon, esophageal and stomach cancers and other gastrointestinal cancers; lung cancers; lymphomas; myelomas; pancreatic cancers; liver cancers; breast cancers; kidney cancers; endocrine cancers; skin cancers; melanomas; angiomas; and brain or central nervous system (CNS) cancers. Tumors or cancers, as defined herein, may be any tumor or cancer, primary or secondary, which is recognized by cytotoxic cells (for example, macrophages) and which induces the tumoricidal effect of the cells upon contact. See, e.g., Alexander and Evans, Nature New Biology 232:76 (1971). Preferred are methods of identifying subjects at risk for tumor-forming cancers, and methods of preventing the same. The term “tumor” is also understood in the art, for example, as an abnormal mass of undifferentiated cells within a multi-cellular organism. Tumors can be malignant or benign. Preferably, the inventive methods disclosed herein are used to identify subjects at risk for developing malignant tumors, and to prevent malignant tumors.

The methods of the present invention are also useful in determining the risk of a subject developing a non-cancer disorder of cell proliferation. These disorders include, but are not limited to hyperplasias, hyperpigmentation of the skin, psoriasis, lipomas, fibroids, keloids, adhesions, and any other disorder wherein cell proliferation is uncontrolled.

These methods are based upon the inventive premise that the measure of cytotoxicity of certain cells (herein described as “diagnostic cells”) of the subject for target cancer cells is a reliable predictor of the risk of developing cancer. For example, human monocytes/macrophages can be tested by methods of the present invention to determine a measure of the cytotoxicity of these cells for human tumor cell lines. The measure of cytotoxicity is negatively correlated to risk for developing cancer; that is, a subject whose diagnostic cells exhibit a high cytotoxicity for cancer cells is at a lower risk for the development of cancer than a subject whose diagnostic cells exhibit a lower cytotoxicity for cancer cells.

In the inventive method of the present invention, a sample of diagnostic cells is first obtained from the subject. Obtaining a sample of diagnostic cells from the subject may be carried out by any method known in the art (e.g., isolating cells from blood samples). Diagnostic cells, as used herein, are cells that express A₁ adenosine receptors and include, but are not limited to, monocytes, macrophages, promonocytes, peripheral blood stem cells (PBSC), and hematopoietic stem cells with monocytes and macrophages being preferred.

After being obtained from the subject, the diagnostic cells are then tested for cytotoxicity for target cancer cells, the results of the testing providing a measure of cytotoxicity of the subject's diagnostic cells. The measure of cytotoxicity may be an indirect measure of cytotoxicity. Examples of indirect measures of cytotoxicity include a measure of the number of A₁ adenosine receptors (A₁AR) in the membranes of the diagnostic cells, or a measure of the affinity of the diagnostic cells for A₁ adenosine receptor specific ligands (i.e., a measure of the affinity of the A₁ARs present in the membranes of the diagnostic cells for A₁AR specific ligands), or a measure of the ability of the diagnostic cells (i.e., the A₁ARs present in the membranes of the cells) to bind MCP-1 protein, or a measure of the ability of the diagnostic cells (i.e., the A₁ARs present in the membranes of the cells) to bind annexins. The measure of cytotoxicity may also be a functional measure of cytotoxicity as defined herein, in which case the diagnostic cells are primed and activated prior to being tested for cytotoxicity.

One indirect method of determining the measure of cytotoxicity of diagnostic cells for target cancer cells is evaluating the number of A₁ adenosine receptors in the membranes of the diagnostic cells according to methods known in the art (e.g., by determining Bₘₐₓ using labeled ligand saturation binding techniques, where Bₘₐₓ is an expression of the density or number of A₁ adenosine receptors present in the membranes of the cells). In general, the higher the number and/or density of A₁ adenosine receptors on the diagnostic cells, the greater the measure of cytotoxicity of the diagnostic cells for target cancer cells, and thus the lower the risk of the subject for developing cancer.

A second and more preferred indirect method of determining a measure of cytotoxicity of the diagnostic cells of the subject is evaluating the affinity of the diagnostic cells for A₁ adenosine receptor (AR) ligands. Alternatively, the affinity of the diagnostic cells for MCP-1 protein, or the affinity of the diagnostic cells for annexins, may be evaluated. These measurements of affinity may be carried out using labeled ligand binding measurement techniques known in the art. Ligands may be labeled with radioactive compounds, fluorescent compounds, biotinylated compounds, and the like. These methods of evaluating the affinity of the diagnostic cells include using saturation binding techniques or competitive binding techniques to determine the affinity of the diagnostic cells for A₁AR ligands, or for MCP-1 protein, or for annexins, as expressed by K_{d} (saturation binding experiments) or Kᵢ (competition binding experiments), with the value of Kᵢ and K_{d} being inversely related to affinity (i.e, the lower the Kᵢ or K_{d}, the higher the affinity). In general, the lower the value of the K_{d} or Kᵢ of the diagnostic cell, the greater the measure of cytotoxicity of the diagnostic cell, and the lower the risk of the subject developing cancer.

Alternatively, or additionally, a measure of cytotoxicity of the diagnostic cells may be a functional measure of cytotoxicity. A functional measure of cytotoxicity refers to a measure of at least one indicia of tumoricidal activity (e.g., release of cytotoxins or cytokines by the cells, or the percentage of killed tumor cells by the diagnostic cells) exhibited by the diagnostic cells. In the practice of the present invention, diagnostic cells are primed and activated prior to determining a functional measure of cytotoxicity.

Diagnostic cells are primed by contacting the cells with a priming agent in an amount sufficient to prime the diagnostic cell, and for a time sufficient to prime the diagnostic cell. As used herein, “contacting” a cell with a substance means (a) providing the substance to the environment of the cell (e.g., solution, in vitro culture medium, anatomic fluid or tissue) or (b) applying or providing the substance directly to the surface of the cell, in either case so that the substance comes in contact with the surface of the cell in a manner allowing for biological interactions between the cell and the substance.

As used herein, “priming” of macrophages refers to a treatment which enhances the metabolic burst of macrophages, wherein the metabolic burst (see Hamilton and Adams, Immunol. Today 8:151 (1987)) is increased over that which would occur in the absence of priming. As used herein, “primed” macrophages refers to those that have undergone a priming treatment; “primers” or “priming agents” refer to agents capable of priming macrophages. The diagnostic cells may be primed using any priming agent known in the art, including but not limited to PMA (see, e.g., Leaver, FEMS Microbiol. Immunol. 47:293 (1989); White, J. Biol. Chem. 259:8605 (1984)); lipopolysaccharide (LPS) (see, e.g., Glaser, J. Biol. Chem 265:8659 (1990); Pace, J. Immunol. 126:1863 (1981); Alexander, Nature New Biol. 232:76 (1971)); platelet activating factor (PAF) (see, e.g., Stewart, Immunology 78:152 (1993); Salzer, J. Clin. Invest. 85:1135 (1990)); tumor necrosis factor alpha (TNF_{α}) or thrombin (see, e.g., Stewart, Immunology 78:152 (1993)); f-met-leu-phe (FMLP) (see e.g., Stewart, Immunology 78:152 (1993)); zymosan (Rankin, J. Clin. Invest. 86:1556 (1990); macrophage stimulating factors including granulocyte macrophage colony stimulating factor (GM-CSF); ionomycin (for example in 1 μM amounts); calcium ionophore (such as A 23187, for example in 0.1-10 μM amounts); gamma interferon (IFNτ, for example in 1-150 units/ml amounts) Flebbe, J. Immunol. 145:1505 (1990); supernatants of tumor cells (Hamilton and Adams, Immunology Today 8:151 (1987); Marvin, J. Surg. Res. 63:248 (1996)); heparin (Heinzelmann et al., Ann. Surg. 229:542 (1999), enoxaparin (Id.), or bacterial products from gram positive organisms (see, e.g., Bacterial Endotoxin Lipopolysaccharides, Morrison and Ryan (Eds.) CRC Press, Boca Raton, Fla., 1992; Hamilton and Adams, Immunology Today 8:151 (1987); Loppnow, Methods Enzymol. 236:3 (1994)). Preferred priming conditions for the type of cell to be to be primed may be determined using routine methods known to those in the art.

After being primed, diagnostic cells are activated by contacting the cells with an activating agent in an amount sufficient to activate the diagnostic cell, and for a time sufficient to activate the diagnostic cell. As used herein, “activated” cells are those which possess tumoricidal functions. Suitable activating agents include and are preferably A₁ adenosine receptor agonists, including but not limited to adenosine; cyclohexyladenosine; various N⁶-substituted A₁ adenosine agonists including but not limited to N⁶ cyclopentyladenosine, N⁶ R-phenylisopropyladenosine, 2-chloro N⁶ cyclopentyl adenosine (CCPA), N⁶ (p-sulfophenyl) alkyl and N⁶ sulfoalkyl derivatives of adenosine (such as N⁶-(p-sulfophenyl) adenosine; 1-deaza analogues of adenosine including but not limited to N⁶ cyclopentyl 1-2-chloro-1-deaza adenosine (1-deaza-2-Cl-CPA); N cycloaklyladenosines; N⁶ bicycloalkyladenosines; ribose modified adenosine receptor analogues including but not limited to 3′-deoxy-R-PIA. See, e.g., Conti, Naunyn-Schmiedeberg's Arch. Pharmacol. 348:108 (1993); Trivedi, J. Med. Chem. 32:8 (1989); Jacobsen, J. Med. Chem. 35:4143 (1992); Thedford, Expl. Cell. Biol. 57:53 (1989); Trewyn, Exp. Pharmacol. 28:607 (1979); Fleysher, J. Amer. Chem. Soc. (August 1968); Fleysher, J. Amer. Chem. Soc. November 1969)); cycloalkyladenosines (see e.g., Moos, J. Med. Chem. 28:1383 (1985)); analogs of R-PIA, CHA, and CPA (see, e.g., Cristalli, J. Med. Chem. 31:1179 (1988)). Van der Wenden, J. Med. Chem. 38:4000 (1995); Jacobson, PJM Med. Res. Rev. 12:423 (1992); Daly, J. Med. Chem. 25:197 (1982).

Lipids may optionally be conjugated to the priming agent(s) and/or the activating agent(s) by techniques known in the art, in order to increase the bioavailability and/or the affinity of the priming agent or activating agent for the cell.

After priming and activation, the diagnostic cells are then tested to determine a functional measure of cytotoxicity of the diagnostic cells for target cancer cells. As used herein, the tumoricidal or cytotoxic effects “of” diagnostic cells, or “by” diagnostic cells, or “for” target cancer cells refers to the presence of cellular activities in diagnostic cells that allow the diagnostic cells to have cytotoxic effects on target cancer cells. These effects can be assessed by analyzing the cellular components of the diagnostic cell (e.g., by evaluating an increase in enzymes or factors associated with signal transduction pathways which produce cytotoxic compounds or cytokines), or by evaluating the cytotoxic compounds produced by the diagnostic cell, or by evaluating the tumoricidal effects of the diagnostic cell on an appropriate target cancer cell, as further described herein. “Target cancer cells” include but are not limited to cells from known and described tumor cell lines. Alternatively, red blood cells may be used as target cells to test the cytotoxicity of the diagnostic cells. See Kleinerman et al., Lancet ii: 1102 (1980). In preferred embodiments of the invention, the target cells are of the same species as the subject. For example, if the subject is human, then the diagnostic cells are preferably tested for cytotoxicity against human tumor or cancer cells or human red blood cells.

A functional measure of cytotoxicity of the diagnostic cells may be determined by several methods. In general, a functional measure of cytotoxicity of the diagnostic cells is determined by testing the diagnostic cells using at least one of several alternate methods of measuring the ability of the diagnostic cells to kill cancer or tumor cells, as indicated by at least one of several indicia. These methods of testing are generally known in the art. For example, one method of determining a functional measure of cytotoxicity useful in the present invention is by calculating or evaluating the percentage of tumor cells killed by primed and activated diagnostic cells of the present invention. The tumor cells may be obtained from known tumor cell lines. In this method, the primed and activated diagnostic cells of the present invention are contacted with target cancer cells under conditions known in the art. After contact with the primed and activated diagnostic cells, the percentage of tumor cells in the sample killed by the primed and activated diagnostic cells is calculated according to techniques known in the art. The percentage of tumor cells killed is then compared to a standard or reference percentage of tumor cell killing in order to determine if the subject is at risk for developing cancer. Such a standard percentage may differ from determination to determination based on kind of tumor cell tested in the sample and other factors, and may change with time based on the accumulation of additional data with regard to the statistical analysis of the development of cancer in a population, but will generally be known to the medical practitioner. In one preferred embodiment of the invention, a percentage of tumor cell killing by the primed and activated diagnostic cells of less than about 10% indicates that the subject is at risk for developing cancer.

Another preferred method of determining a functional measure of cytotoxicity is evaluating the concentration of A₁ adenosine receptor ligand required to produce cytotoxic or tumoricidal functional effects in the diagnostic cells, which functional effects include but are not limited to the release of cytokines or cytotoxic substances (e.g., TNF-α, interleukin-1, or thromboxane) by the diagnostic cells. In general, the lower the concentration of A₁AR ligand required to produce a tumoricidal effect in the cell, the greater the measure of cytotoxicity of the diagnostic cells, and the lower the risk of the subject developing cancer. Alternatively, the amount of cytokine released by the primed and activated diagnostic cells can be measured and correlated to risk of developing cancer. In general, the greater the amount of cytokine released by the diagnostic cells, the greater the measure of cytotoxicity of the diagnostic cells, and the lower the risk of the subject developing cancer.

Other methods of determining a functional measure of cytotoxicity include, but are not limited to methods of evaluating the ability and efficiency of the A₁ adenosine receptors of the diagnostic cells to couple to signal-transduction related pathways such as G-proteins, protein kinase C, phospholipase C, phospholipase D, phospholipase A₂, or NF-κB, according to techniques that are known to those skilled in the art.

As will be evident from the foregoing, the measure of cytotoxicity of diagnostic cells may be determined, and preferably is determined by using more than one test for cytotoxicity. For example, a medical practitioner may determine a measure of cytotoxicity of the diagnostic cells of a subject both by calculating the percentage of tumor cells killed by the diagnostic cells (a functional measure of cytotoxicity), and by determining the affinity of the diagnostic cells for A₁ AR ligands (an indirect measure of cytotoxicity). The combination of results of these two tests thus provides the practitioner with more than one indicia of whether or not the subject is at risk for developing cancer. Whether or not a subject is determined to be at risk for developing cancer may be determined by the practitioner by comparing the results of the tests for cytotoxicity with standards correlating a measure of cytotoxicity with the risk of developing cancer.

The measure of cytotoxicity of the diagnostic cells positively correlates with the likelihood of successful prevention of cancer in the subject. Once a subject is identified as being “at risk” for developing cancer, inventive methods of the present invention may be used to prevent cancer in the subject. Although these methods may be applied to subjects identified as being at risk by the methods of the present invention, subjects that are determined to be at risk for developing cancer as identified by other methods of identifying or assessing risk of cancer are also suitable subjects of the preventative and diagnostic methods of the present invention. Additionally, “at risk” subjects may also be subjects that are defined as such because of other indicia of cancer risk. For example, suitable subjects may include, but are not limited to, individuals with a family history of cancer, individuals who have previously been treated for cancer, individuals who have been exposed to carcinogens (e.g., heavy smokers), individuals exposed to medications or medical treatments associated with the development of cancer (e.g., estrogens or radiation therapy), individuals determined to have an increased likelihood of developing cancer by genetic testing, and individuals presenting any other clinical indicia suggesting that they have an increased likelihood of developing cancer. In particular, subjects who have been previously treated for cancer by known methods (e.g., radiation, chemotherapy) and who are deemed to be essentially “cancer-free” are suitable subjects for the preventative and diagnostic methods of the present invention.

By the terms “prevention of cancer” or “preventing cancer” it is intended that the inventive methods eliminate or reduce the incidence or onset of cancer, as compared to that which would occur in the absence of treatment. Alternatively stated, the present methods slow, delay, control, or decrease the likelihood or probability of cancer in the subject, as compared to that which would occur in the absence of treatment. It is specifically intended that the term “prevention of cancer” includes the prevention of the recurrence of cancer in a subject who has previously been diagnosed with cancer and successfully treated. One therapeutic goal of the preventative methods of the present invention is to increase the cytotoxicity of the macrophages, monocytes, promonocytes and peripheral blood stem cells of the subject until these cells are sufficiently cytotoxic for target cancer cells such that cancer does not develop in the subject. In one embodiment of the preventative methods of the invention, the cytotoxicity of the macrophages and/or monocytes of the subject is increased such that a functional measurement of the percentage of tumor cells killed (as set forth in the description of diagnostic methods herein) by the macrophages and monocytes is at least about 30%.

The preventative methods of the present invention utilize the cytotoxic effects of primed and activated cells (including but not limited to macrophages, monocytes, promonocytes and peripheral blood stem cells) to prevent the growth of tumor cells. In essence, the preventative methods of the present invention are useful in “arming” the cells of a subject at risk for cancer by increasing the cytotoxicity of the cells of the subject for target cancer cells. Preventative methods of the present invention may be, and preferably are carried out in vivo. Increasing the cytotoxicity of the cells of the subject may be carried out by one or more of several methods. For example, the present methods may be carried out by direct activation of the cells of the subject by administering to the subject priming agents and/or A₁ adenosine receptor agonists (activating agents), either systemically or locally (i.e., directly to the cells of the subject). In one embodiment of the invention, a subject determined to be at risk for developing cancer is systemically administered at least one priming agent, as described herein. The priming agent may be administered to the subject by any of the accepted methods of administering pharmaceutical compounds to a subject, including but not limited to administration parenterally (i.e, intravenously, subcutaneously, intradermally, intramuscularly, intraarticularly); orally, rectally, topically, (including buccal, sublingual, dermal and intraocular administration), transdermally and by inhalation. The dosage of the priming agent will be an amount effective to prime the cells (i.e., macrophages, monocytes, promonocytes, peripheral blood stem cells) of the subject and will vary with the compound used and the condition or state (i.e., size, age) of the subject. The daily dose may be divided among one or several unit dose administrations. Treatments may continue on a chronic basis as necessary. The priming agent being administered to the subject may be administered to the subject by liposomal delivery according to methods known in the art, but may also be formulated into any acceptable pharmaceutical formulation (e.g., dissolved in a carrier suitable for injection, admixed with a carrier in tablet or capsule form).

After administering a priming agent to the subject determined to be at risk of developing cancer, the cytotoxicity of the diagnostic cells of the subject (i.e., macrophages and/or monocytes) may be evaluated according to the diagnostic methods of the invention set forth herein. Should the practitioner determine, using these diagnostic methods, that a further increase in cytotoxicity of the subjects macrophages, monocytes, promonocytes or peripheral blood stem cells is desired, then the present invention also provides for the administration of an activating agent (i.e., an A₁ adenosine receptor agonist) in addition to the administration of a priming agent. The activating agent may be administered in conjunction with a priming agent of the present invention, or separately. The activating agent may be administered in essentially the same manner as the priming agent, as set forth herein.

In one embodiment of the invention, a priming agent and an activating agent are formulated together (i.e., encapsulated in) in a liposomal formulation according to techniques known in the art, and then administered concurrently to the subject. See, e.g., U.S. Pat. Nos. 5,527,528 to Allen et al.; U.S. Pat. No. 5,013,556 to Woodle et al., U.S. Pat. No. 5,882,679 to Needham; and U.S. Pat. No. 5,766,627 to Samkaram et al, the disclosures of which are incorporated herein by reference. In a preferred embodiment of the invention, the liposomal formulation comprising the priming agent and the activating agent is formulated in a timed-release formulation such that the priming agent is released prior to the release of the activating agent.

Preventative methods of the present invention also include subjecting certain cells (e.g., macrophages, monocytes, promonocytes, peripheral blood stem cells) of the subject with a number of different treatments to increase the expression of the human A₁ adenosine receptor and/or to increase the affinity of these receptors for A₁ adenosine receptor ligands. These methods may be carried out in accordance with the methods described in co-pending and co-owned U.S. patent application Ser. No. 08/748,559, filed Nov. 8, 1996, the disclosure of which is incorporated herein in its entirety. Preventative methods that increase the expression of human A₁ adenosine receptors in cells such as human monocytes, macrophages, promonocytes and peripheral blood stem cells include: transfecting these cells with cDNA for the human A₁ adenosine receptor gene; treating these cells with drugs, including chemotherapeutic drugs, cisplatin, daunorubicin, doxorubicin, or mitoxantrone, dexamethasone, or other drugs such as carbamazepine; treating the cells with adenosine receptor antagonists (e.g., theophylline); and/or subjecting the cells to ischemic conditions. See U.S. Pat. No. 5,320,962 to Stiles et al., Nie, et al., Mol. Pharmacol. 53:663 (1998); Gerwins and Fredholm, Mol. Pharmacol. 40:149 (1991); Lupica et al. Synapse 9:95 (1991); Ren and Stiles, Mol. Pharmacol. 55:309 (1999); Biber et al., Neuropsych. Pharmacol. 20:271 (1999).

Methods of preventing cancer in subjects deemed at risk for developing cancer include methods that increase the affinity of A₁ adenosine receptors (expressed in macrophages and monocytes, for example) for A₁ adenosine receptor ligands. These methods of increasing the affinity of A₁ adenosine receptors for A₁ adenosine receptor ligands include contacting the cells expressing A₁ adenosine receptors with allosteric enhancers for A₁ adenosine receptors, such as PD 81,723 which increases the affinity and binding of an A₁ adenosine receptor ligand for A₁ adenosine receptors and coupling of the receptor to the G protein; contacting the cells with divalent cations, including magnesium and calcium; and/or contacting the cells with adenosine deaminase, or immunomodulators or priming agents, such as lymphokines, MDP, MTP, MTP-PE, IFN-γ, PMA, GM-CSF, or FMLP. These treatments may be given in vitro, in vivo, or in vivo, may be delivered by liposomes, and may be used in combination with treatments to increase A₁ adenosine receptor expression as described herein.

Cells (e.g., macrophages, monocytes, promonocytes, peripheral blood stem cells) of the at-risk subject in whom prevention of cancer is desired may also be treated with 1,25-dihydroxycholecalciferol to improve terminal maturation of monocyte/macrophage cultures, and/or M-CSF or GM-CSF to stimulate the production of these cells. Bartholeyns, Res. Immunol. 144:288 (1993). These treatments may be used in combination with treatments to increase A₁ adenosine receptor expression or binding of A₁ adenosine receptor ligands to these cells. Also, promonocytes obtained from the bone marrow or from peripheral blood hematopoietic stem cells of the subject, or from commercial sources for human monocytes and human macrophages, may be tested for A₁ adenosine receptor expression and affinity. These cells may also be transfected with cDNA for human A₁ AR gene, or treated with other drugs, including chemotherapeutic drugs, dexamethasone, adenosine receptor antagonists, or subjected to ischemic conditions in order to increase A₁ AR expression. These cells may also be treated with allosteric enhancers for A₁ adenosine receptors, divalent cations, adenosine deaminase, or immunomodulators or priming agents, as described above. See, e.g, Bhattacharya and Linder Biochimica and Biophys. Acta 1265:15 (1995); Musser et al., J. Pharmcol. Exp. Ther. 288:446 (1999). In addition, promonocytes, monocytes, peripheral blood stem cells, or macrophages of the subject may be transfected with cDNA for human MCP-1 protein or other cDNA for other human genes such as TNF-α or other immunomodulator or priming agents, or adhesion molecules such as CD11/CD18, or ICAM-1, which promote hematopoietic growth, maturation, function, or survival in combination with cDNA for human A₁ adenosine receptor. These treatments may induce these promonocytes, peripheral blood stem cells, monocytes, and macrophages to be more efficient and have greater cytotoxic effects towards tumor cells and reduce the number of macrophages or the macrophage-to-tumor cell ratio necessary for an adequate tumoricidal effect for these cells for the prevention of developing cancers in vivo by recognizing and destroying tumor cells and preventing tumor growth.

Finally, monocytes, macrophages, promonocytes and/or peripheral blood stem cells may be obtained from the subject or other sources (i.e., from other subjects, or from known cell lines, or from commercial sources), cloned to increase A₁ adenosine receptor expression, function and tumoricidal effects, and then administered to the subject either systemically or locally. In a preferred embodiment of the invention, cells treated according to the methods described herein may be administered to the subject in whom cancer prevention is desired according to the administration techniques set forth in co-pending and co-owned application Ser. No 09/748,559, incorporated herewith in its entirety.

Kits for determining a subject's risk of developing cancer will include at least one container sized to house at least one reagent useful in determining a measure of cytotoxicity of diagnostic cells as defined herein, and printed instructions for assessing whether or not a subject is at risk for developing cancer. Kits for preventing cancer in a subject will include at least one container sized to house at least one reagent useful in preventing cancer according to methods described herein, as well as printed instructions for carrying out these methods. As used herein, the term “reagent” means any compound, composition or biological agent (i.e., samples, aliquots or “doses” of cells, cDNAs, recombinant DNAs, isolated genes, antibodies, etc.) useful in carrying out any method of the present invention, including but not limited to priming agents, activating agents, A₁ adenosine receptor ligands (including agonists, antagonists and antibodies to A₁ adenosine receptors), antibodies and ligands for MCP-1 protein, antibodies and ligands for annexins, antibodies and ligands for cytokines and cytotoxic compounds produced by cells, cDNAs encoding A₁ adenosine receptors and compounds useful in transfecting the cDNAs into cells, drugs and other compounds for increasing A₁ adenosine receptor expression, drugs and other compounds for increasing affinity of cells for A₁ adenosine receptors, drugs and other compounds for stimulating the production of monocytes and macrophages and/or the terminal differentiation thereof, buffers and carriers useful in isolating and preparing cells and/or membranes for analysis and treatment, buffers and carriers useful in carrying out saturation and competition binding assays, allosteric enhancers for A₁ adenosine receptors, known anti-cancer therapeutic compounds, and radioactive and non-radioactive labeling compounds.

A diagnostic kit of the present invention may include reagents for performing indirect determinations of cytotoxicity of diagnostic cells (i.e., reagents for performing tests measuring the number of A₁ adenosine receptors in the membranes of diagnostic cells, and/or reagents useful in performing tests measuring the affinity of diagnostic cells for A₁ adenosine receptor ligands and/or for MCP-1 protein). Accordingly, a diagnostic kit of the present invention may comprise a container containing at least one ligand for A₁ adenosine receptors, including antibodies for the A₁ adenosine receptor, A₁ adenosine receptor agonists, and A₁ adenosine receptor antagonists. These reagents may be labeled with radioactivity or a non-radioactive label (i.e., fluorescent label, biotinylated label), and are useful in measuring the Kᵢ of these ligands for A₁ adenosine receptors in a competition assay. A diagnostic kit may alternatively or additionally include a container comprising at least one ligand for MCP-1 or annexins, including antibodies for MCP-1 or annexins. These reagents are useful in determining the Kᵢ of these ligands for A₁ adenosine receptors in the membranes of a subject's diagnostic cells (e.g., in a competition assay). Diagnostic kits for determining indirect measures of cytotoxicity of diagnostic cells will include printed instructions for conducting the appropriate tests, and may include instructions for isolating diagnostic cells from a subject, instructions for isolating and affixing to a solid support membranes from the diagnostic cells for use in the tests, and instructions for conducting the tests themselves. The printed instructions will also include instructions for correlating the results of the tests with the risk of the subject developing cancer.

A diagnostic kit of the present invention may include reagents for performing functional determinations of cytotoxicity of diagnostic cells (i.e., reagents for performing tests measuring cytokine release from primed and activated diagnostic cells, and/or reagents for performing tests of tumor cell killing by primed and activated cells). Accordingly, a diagnostic kit of the present invention may include at least one container sufficiently sized to include at least one priming agent, at least one activating agent, and at least one reagent for determining a functional measure of cytotoxicity of diagnostic cells of a subject. If the functional measure of cytotoxicity is the percent of tumor cells killed by the primed and activated diagnostic cells of the subject, then the kit may contain a sample of target cancer cells (e.g., tumor cells) that the primed and activated diagnostic cells of the subject may be tested against. Diagnostic kits for determining functional measures of cytotoxicity of diagnostic cells will include printed instructions for conducting the appropriate tests, and may include instructions for isolating diagnostic cells from a subject, and instructions for conducting the tests themselves. The printed instructions will also include instructions for correlating the results of the tests with the risk of the subject developing cancer.

Kits useful for the prevention of cancer according to the preventative methods of the present invention will include at least one container sufficiently sized to include at least one reagent for increasing A₁ adenosine receptor expression in cells (i.e., macrophages, monocytes, peripheral blood stem cells, promonocytes) of an at-risk patient, and/or at least one reagent for increasing binding of A₁ adenosine receptor ligands to these cells, and/or at least one reagent for directly priming and/or activating cells of the subject in vitro or in vivo. A kit useful for prevention of cancer according to the preventative methods of the present invention may alternatively or additionally include at least one sample (or dose) of macrophages, monocytes, promonocytes or peripheral blood stem cells cloned or treated with, for example, cDNA encoding A₁ adenosine receptors, drugs or ischemia in order to increase A₁ adenosine receptor expression, function and tumoricidal effect. Prevention kits of the present invention will include printed instructions for administering to at-risk subjects the reagent or reagents useful in preventing cancer.

The following Examples are provided to illustrate the present invention, and should not be construed as limiting thereof.

### EXAMPLE 1

### Preparation of Mouse Macrophage Cell Culture

Endotoxin-sensitive (LPS-sensitive) mouse macrophages (ATCC IC-21) are grown to confluency in RPMI medium containing 2 mM glutamine, 1.5 g/L sodium bicarbonate, 4.5 g/L glucose, 10 mM HEPES, 1.0 mM sodium pyruvate and 10% fetal bovine serum in 24-well culture plates or 75 mm² culture flasks in a humidified CO₂ incubator.

Endotoxin-resistant (LPS-resistant) mouse macrophages (ATCC WB264-9C) are grown to confluency in minimum essential medium (Eagle) with Earle's balanced salt solution containing 10% fetal bovine serum in 24-well culture plates or 75 mm² culture flasks in a humidified CO₂ incubator.

### EXAMPLE 2

### In vitro Cytotoxicity Experiments

IC-21 macrophages, WBC264-9C macrophages, and WBC264-9C macrophages previously treated with cisplatin (2.5 μM for 24 hours) are incubated with serum free media for 24 hours before the experiment.

B-16 melanoma cells are washed with serum free RPMI medium by centrifugation. 25-30 μCi of Na ⁵¹Cr is diluted to 1 ml of RPMI and transferred to the melanoma cells suspension. The melanoma cells are incubated with Na ⁵¹Cr at 37° C. for 1 hour. The unincorporated radioactivity is removed by centrifugation at 1000×g for 10 minutes. The melanoma cells are allowed to leak for 2 hours and then washed again. The final suspension is diluted in RPMI. The cell number is counted in a hemocytometer.

Macrophages are washed with medium containing 100 mM MgCl₂, 2 U/ml adenosine deaminase and 10% fetal bovine serum. Macrophages are then challenged with 1 μM PMA in the above medium for 2 hours and then stimulated with different concentrations of (0-1 μM) of 2-chloro-N⁶-cyclopentyl adenosine (CCPA) for 30 minutes. The macrophages are then incubated with ⁵¹Cr-labelled B-16 melanoma cells at a ratio of 20:1. Six hours later, a fraction of the supernatant is aspirated and counted in a gamma counter.

Spontaneous release of the radioactivity is measured from the wells that do not contain macrophages. Total releasable counts are measured by lysing the melanoma cells in hypotonic medium.

The results are expressed as percentage (%) ⁵¹Cr release as follows:

$$percentage {( \% )}^{51} Cr release = \frac{Experimental release - spontaneous release}{Total releasable counts - spontaneous release}$$

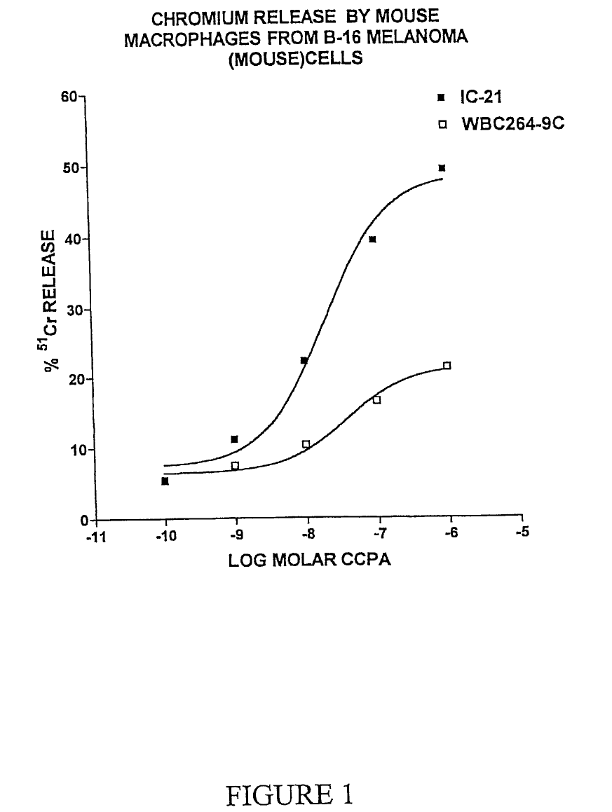

Percentage ⁵¹ Cr release is a measure of the lysing of cells (i.e., killing of cells), and is therefore an indicia of cytotoxicity. The results of this experiment are illustrated in FIG. 1, in which percentage ⁵¹Cr release is expressed as a function of increasing log molar CCPA, and in which filled-in squares represent IC-21 macrophage data and open squares represent WBC264-9C macrophage data.

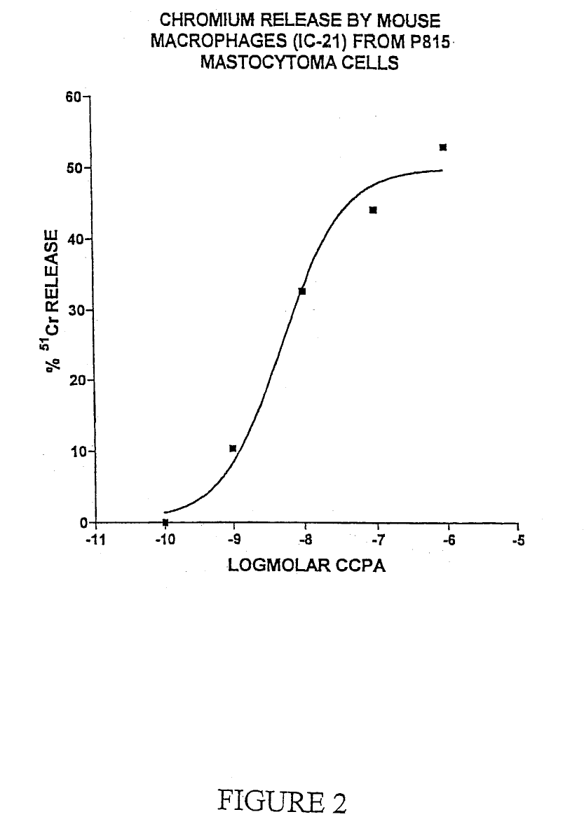

This same protocol is repeated, except that P815 mouse mastocytoma tumor cells are used instead of the B-16 melanoma cells. The results of this experiment are shown in FIG. 2. As illustrated by the results of these experiments, primed and activated macrophages from LPS-sensitive animals exhibit a significantly higher cytotoxicity against tumor cells when compared to the cytotoxicity of primed and activated macrophages from LPS-resistant animals.

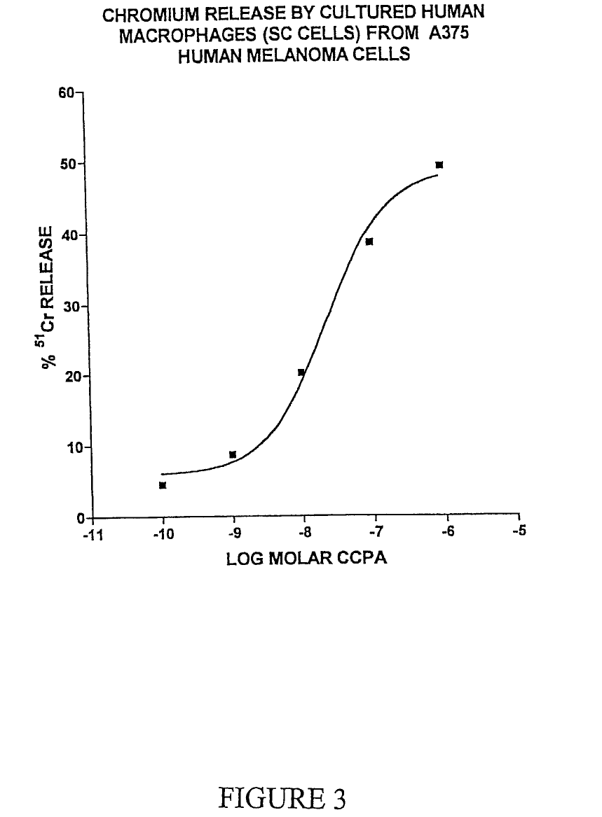

The protocol is also repeated using cultured human macrophages (SC cells) and A375 human melanoma cells. The results of this experiment are shown in FIG. 3.

### EXAMPLE 3

### Preparation of Mouse Macrophage Membranes

The macrophages described in Example 1 are lysed in 10 mM Tris HCl buffer, pH 7.4, containing 5 mM EDTA, 10 μg/ml soybean trypsin inhibitor, 10 μg/ml benzamidine and 2 μg/ml pepstatin. The macrophages are homogenized using a cell sonicator to produce a homogenate. The homogenate is centrifuged at 1000×g for 10 minutes. The supernatant is centrifuged again at 40,000×g for 20-30 minutes at 4° C. The membrane pellet is resuspended in 50 mM Tris-HCl buffer, pH 7.4, 10 mM MgCl₂, 1 mM EDTA, 10 μg/ml soybean trypsin inhibitor, 10 μg/ml benzamidine, 2 μg/ml pepstatin and 5 U/ml adenosine deaminase.

### EXAMPLE 4

### Saturation Binding Experiments

Saturation binding experiments are performed in membrane fractions (10-20 μg protein) from IC-21 macrophages, WBC264-9C macrophages, and WBC264-9C macrophages previously treated with cisplatin (2.5 μM for 24 hours). Incubation of the macrophage fractions with the A₁ adenosine receptor ligand ¹²⁵I-BWA844U (see Patel et al., J. Pharmacol. Exp. Ther. 33:585 (1988) in concentrations ranging from 1.25-20 nM was carried out at 37° C. for 1 hour. Non-specific binding is determined in the presence of 1 mM theophylline. Following incubation, membrane bound radioactivity is separated by filtration over a cell harvester and counted in a gamma counter. The data is analyzed by non-linear regression using GraphPad Prism software. Thc Bₘₐₓ and k_{d} values are calculated from the analyzed data.

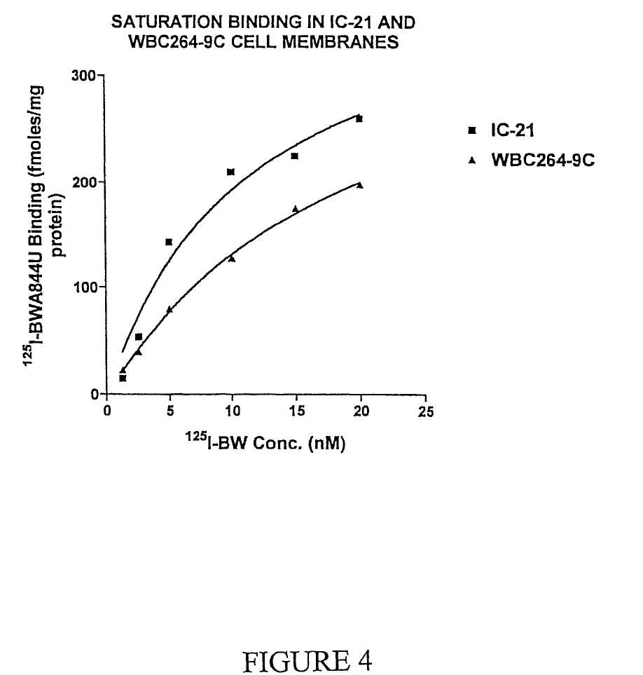

Results of this experiment are shown in FIG. 4, in which Bₘₐₓ (expressed in units of fmol/mg protein) is plotted as a function of increasing concentration ¹²⁵I-BWA844U. Solid squares represent data from IC-21 macrophage membranes, while solid triangles represent data from WBC264-9C macrophage membranes. These results illustrate macrophage membranes from LPS-sensitive mice bind more A₁ adenosine receptor ligand than do macrophage membranes from LPS-resistant mice.

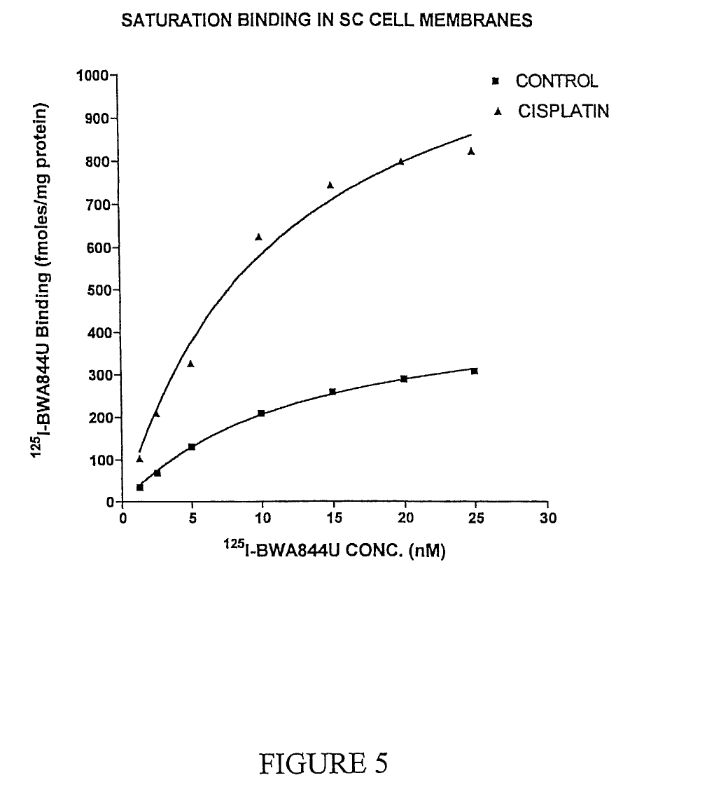

This same protocol is repeated using membranes from human macrophages (SC cells). The solid squares represent data from membranes not treated with cisplatin, while solid triangles represent data from membranes treated with cisplatin (2.5 μM for 24 hours). The results of this experiment are shown in FIG. 5. These results show that pre-treating macrophage membranes with cisplatin increased the Bₘₐₓ of the macrophage for the A₁ adenosine receptor ligand by approximately three-fold.

### EXAMPLE 5

### Competition Experiments with Human Recombinant MCP Protein

Competition experiments are performed with different concentrations of human recombinant MCP-1 protein with a fixed concentration (0.1-0.2 nM) of ¹²⁵I-BWA844U in macrophage membranes from IC-21 macrophages, WBC264-9C macrophages, and WBC264-9C macrophages previously treated with cisplatin (2.5 μM for 24 hours). Non-specific binding is determined in presence of 1 mM theophylline. Membrane bound radioactivity is separated by filtration over a cell harvester. The data is analyzed by non-linear regression analysis using GraphPad prism software and the Kᵢ value is calculated.

Affinity for MCP-1 is lower (i.e., the Kᵢ is higher) in membranes from WBC264-9C macrophages than it is for membranes from IC-21 macrophages. Pretreatment with cisplatin increases the affinity of membranes from WBC264-9C macrophages for MCP-1.

### EXAMPLE 6

### Functional Experiments: TNF-α Release

IC-21 macrophages, WBC264-9C macrophages, and WBC264-9C macrophages pretreated with cisplatin (2.5 μM for 24 hours) are washed with medium containing 100 mM MgCl₂, 2 U/ml adenosine deaminase and 10% fetal bovine serum. Macrophages are then challenged with 1 μM PMA in the above medium for 2 hours and then stimulated with different concentrations of (0-1 μM) of 2-chloro-N⁶-cyclopentyl adenosine (CCPA) at 37° C. for 1-6 hours. The supernatant is collected and the TNF-α levels are estimated by commercially available ELISA kits (R & D Systems, Minneapolis, Minn.).

Following priming with PMA and activation with CCPA, TNF-α release is lower in WBC264-9C macrophages than it is in IC-21 macrophages. Pretreatment of the WBC264-9C macrophages with cisplatin increases TNF-α release from WBC264-9C macrophages.

### EXAMPLE 7

### In vivo Experiments for Prevention of B-16 Mouse Melanomas with LPS-Sensitive and LPS-Resistant Macrophages

Mice, tumors: Mice are implanted subcutaneously by injection with 1×10⁵ mouse B-16 melanoma cells in the flank. Animals are sorted into the various treatment groups (see Table 1), and treatments are initiated 30 minutes prior to injections of B-16 melanoma tumor cells. Estimated tumor weight is calculated using the formula:

$$Tumor Weight ( mg ) = \frac{{w}^{2} \times l}{2}$$

Where w width and l=length in mm of B-16 melanoma

Treatment: The treatment plan for animals in this study is shown in Table 1, below. Mice are sorted on Day 1 into nine groups with ten animals per group. Dosing via intravenous (TV) tail vein injection or intraperitoneal (IP) is initiated on Day 1 for all agents. Mice are dosed with IV treatments 4 times a week for 4 weeks (qd×4×4 wk). Mice arc dosed with IP treatments once a day for 5 days (qd×5). Tumor caliper measurements are obtained twice weekly for all mice. Animals are weighed twice weekly. The study is terminated on Day 60.

Endpoint: The tumor growth delay (TGD) method is used in these experiments. In the TGD method, each animal is euthanized when its B-16 melanoma reaches a size of 2.0 g; this is considered a cancer death. Mean Day of Survival (MDS) values are calculated for all groups. Treatment-effected mean increases in survival of various groups are compared to each other and to the mean survival times of control tumor-bearing mice (no treatment) and tumor-bearing mice receiving only vehicle (PBS) or tumor bearing mice receiving only naïve macrophages.

$$Time to endpoint ( calculated ) = Time to exceed endpoint ( observed ) - \frac{{Wt}_{2} - endpoint weight}{\frac{{Wt}_{2} - {Wt}_{1}}{{D}_{2} - {D}_{1}}}$$

where:

Time to exceed endpoint (observed)=number of days it takes for each tumor to grow past the endpoint (cut off) size. This is the day the animal is euthanized as a cancer death.

D₂=day animal is euthanized.

D₁=last day of caliper measurement before tumor reaches the endpoint.

Wt₂=tumor weight (mg) on D₂

Wt₁=tumor weight (mg) on D₁

Endpoint weight=predetermined “cut-off” tumor size for the model being used.

As shown in Table 1, below, treatment according to the above protocol causes either complete tumor prevention or partial tumor prevention in an animal. Also, therapy may prevent a neoplasm's growth to a small size but not reach the 2.0 g cut-off. Specifically, there is complete or partial prevention of tumor growth in animals pretreated with primed and activated LPS-sensitive macrophages (IC-21). There is less tumor growth prevention in animals pretreated with primed and activated LPS-resistant macrophages (WBC264-9C) than in the animals pretreated with primed and activated LPS-sensitive macrophages (IC-21). Tumor growth prevention in animals pretreated with primed and activated LPS-resistant macrophages pretreated in vitro with cisplatin (2.5 μM for 24 hours) (WBC264-9C+cisplatin) is greater than that observed in animals pretreated with primed and activated LPS-resistant macrophages (WBC264-9C) without cisplatin in vitro pretreatment or cisplatin administered intraperitoneally alone. There is complete or partial tumor prevention in animals treated with naïve (without priming or activation) LPS-resistant macrophages pretreated in vitro with cisplatin (2.5 μM for 24 hours) (WBC264-9C+cisplatin).

Toxicity: Animals are weighed twice weekly during the study. Mice are examined several times a week for clinical signs of drug-related side effects.

Statistics: The unpaired t-test and Mann-Whitney test (analyzing means and medians, respectively) are used to determine the statistical significance of any difference in survival times between a treatment group and the control group. All statistical analyses are conducted at p level of 0.05 (two-tailed). P-values obtained dictate the percent chance that random sampling from identical populations would lead to a difference larger than observed (p=0.03=3% chance). Prism (GraphPad) version 3a is used for all statistical analysis and graphs.

TABLE 1

| Treatment Groups | | | | | |
|---|---|---|---|---|---|
| Group No. | n | Drug/Agent | Dose | Route | Schedule |
| 1 | 10 | No treatment | | | |
| 2 | 10 | PBS | 0.2 ml | IV | qd × 4 × wk |
| 3 | 10 | Naïve Macro/IC-21 | 1 × 10⁷ | IV | qd × 4 × 4 wk |
| 4 | 10 | Naïve Macro/WBC264-9C | 1 × 10⁷ | IV | qd × 4 × 4 wk |
| 5 | 10 | Naïve Macro/WBC264-9C + | 1 × 10⁷ | IV | qd × 4 × 4 wk |
| | | Cisplatin | 1 × 10⁷ | IV | qd × 4 × 4 wk |
| 6 | 10 | P/AMacro/IC-21 | 1 × 10⁷ | IV | qd × 4 × 4 wk |
| 7 | 10 | P/AMacro/WBC264-9C | 1 × 10⁷ | IV | qd × 4 × 4 wk |
| 8 | 10 | P/AMacro/WBC264-9C + | 1 × 10⁷ | IV | qd × 4 × 4 wk |
| | | Cisplatin | | | |
| 9 | 10 | Cisplatin | 100 mg/m² | IP | qd × 5 |
| Table 1 Legend: Macrophages (Macro) from LPS-sensitive mice (IC-21), LPS-resistant mice (WBC264-9C), and macrophages previously treated in vitro with cisplatin (2.5 μM for 24 hours) from LPS-resistant mice (WBC264-9C + cisplatin) are washed with medium containing 100 nM MgCl₂,2 U/ml adenosine deaminase and 10% fetal bovine serum. These macrophages are then primed (P) with PMA (1 μM) in this medium for 2 hours and then activated (A) with CCPA (1 μM) for 1 hour. Naïve | | | | | |
| #macrophages are not treated with PMA and CCPA. Macrophages are injected intravenously (IV) in 0.2 ml PBS. | | | | | |

The foregoing is illustrative of the present invention and is not to be construed as limiting thereof. The invention is defined by the following claims, with equivalents of the claims to be included therein.

That which is claimed is:

1. A method of determining a subject's risk for developing cancer, comprising:
obtaining a sample of diagnostic cells from a subject, said diagnostic cells selected from the group consisting of macrophages, monocytes, promonocytes and peripheral blood cells; and then
determining a measure of cytotoxicity of the diagnostic cells for target cancer cells, the measure of cytotoxicity correlating negatively with the risk for developing cancer; whereby said subject's risk for developing cancer is determined.

2. The method according to claim 1, wherein the measure of cytotoxicity is determined by evaluating the affinity of the diagnostic cells for at least one A₁ adenosine receptor ligand.

3. The method according to claim 1, wherein the measure of cytotoxicity is determined by evaluating the number of A₁ adenosine receptors on the diagnostic cells.

4. The method according to claim 1, wherein the measure of cytotoxicity is determined by evaluating the affinity of the diagnostic cells for MCP-1 protein.

5. The method according to claim 1, further comprising the steps of:
priming the diagnostic cells by contacting the diagnostic cells with a priming agent in an amount sufficient to prime the diagnostic cells; and
activating the diagnostic cells by contacting the diagnostic cells with an activating agent in an amount sufficient to induce cytotoxicity in the diagnostic cells;
wherein the priming and activating steps occur prior to determining the measure of cytotoxicity of the diagnostic cells for target cancer cells.

6. The method according to claim 5, wherein the measure of cytotoxicity is determined by evaluating the release of cytotoxins from the diagnostic cells.

7. The method according to claim 5, wherein the cytotoxin is tumor necrosis factor α (TNF-α.

8. The method according to claim 5, wherein the measure of cytotoxicity is determined by evaluating the percentage of target cancer cells killed by the diagnostic cells.

9. The method according to claim 5, wherein the activating agent is an A₁ adenosine receptor agonist.

10. The method according to claim 5, wherein the activating agent is conjugated to a lipid.

11. The method according to claim 5, wherein said priming agent is selected from the group consisting of phorbol myristoyl acetate (PMA), lipopolysaccharide (LPS), interferon gamma (IFNτ), granulocyte-macrophage colony stimulating factor (GMCSF), and f-met-leu-phe (fMLP).

12. The method according to claim 5, wherein said priming agent is conjugated to a lipid.

13. The method according to claim 1, wherein said subject is human.

14. The method according to claim 5, wherein said measure of cytotoxicity is determined by evaluating the affinity of the diagnostic cells for at least one A₁ adenosine receptor ligand and by evaluating the percentage of target cancer cells killed by the diagnostic cells.
